(19) United States
(12) Patent Application Publication
(10) Pub. No.: US 2026/0166107 A1
(43) Pub. Date: Jun. 18, 2026

(54) COMPOSITION FOR SUPPORTING A CARDIOVASCULAR AND IMMUNE SYSTEM AND METHOD OF USE

(71) Applicant: DADFUEL LLC, Boca Raton, FL (US)

(72) Inventor: Brandon Bal

(21) Appl. No.: 18/982,353

(22) Filed: Dec. 16, 2024

(51) Int. Cl. A61K 36/68; A61K 31/05; A61K 31/07; A61K 31/23; A61K 31/355; A61K 31/375; A61K 31/593; A61K 33/04; A61K 33/30; A61K 36/062; A61K 36/48; A61K 36/61; A61K 36/81; A61K 36/899

(52) U.S. Cl. CPC A61K 36/68; A61K 31/05; A61K 31/07; A61K 31/23; A61K 31/355; A61K 31/375; A61K 31/593; A61K 33/04; A61K 33/30; A61K 36/062; A61K 36/48; A61K 36/61; A61K 36/81; A61K 36/899; A61K 2236/19

(57) ABSTRACT

A composition that includes one or more plant-based ingredients, natural extracts, polyphenolic compounds, yeast, vitamins, minerals, and fats to provide a composition generally intended to be administered orally to support and promote a healthy heart, cardiovascular system, and immune system. The composition may comprise, in one exemplary embodiment, a combination of psyllium, tomato extract, fenugreek extract, resveratrol, red yeast rice extract, and medium chain triglyceride (MCT) oil, vitamins C, D3, E, and A, zinc, selenium (as selenomethionine), and Myrciaria dubai or organic camu camu fruit powder in various amounts.

### FIELD OF THE INVENTION

The present invention relates generally to compositions and, more particularly, to a composition designed to support and maintain a healthy cardiovascular and immune system and method of use of the same.

### DETAILED DESCRIPTION

All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary, or the following detailed description. It is also to be understood that the specific devices and processes described in the following specification, are simply exemplary embodiments of the inventive concepts defined in the appended claims. Hence, specific compositions relating to the embodiments disclosed herein are not to be considered as limiting, unless the claims expressly state otherwise. The terminology used herein is for the purpose of describing particular embodiments only and is not intended to be limiting of the invention. As used herein, the indefinite articles “a”, “an” and “the” should be understood to include plural reference unless the context clearly indicates otherwise. The phrase “and/or,” as used herein, should be understood to mean “either or both” of the elements so conjoined, i.e., elements that are conjunctively present in some cases and disjunctively present in other cases.

The term “about” used herein indicates plus or minus +/− a reasonably acceptable percentage amount under the applicable manufacturing standard to accurately reflect the actual nutrient content.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

The term “effective amount,” as used with reference to the present composition as described herein, refers to the quantity of the composition or ingredients thereof necessary to render the desired therapeutic or effective result. For example, an effective amount is a level effective to support a subject's immune system or cardiovascular system. Amounts effective for the particular therapeutic goal sought will depend upon a variety of factors including: the disorder being treated and its severity; the bioavailability, and activity of the specific compound, agent, biologic or pharmaceutical composition used; the route or method of administration and introduction site on the subject; the rate of clearance of the specific compound or biologic and other pharmacokinetic properties; the duration of treatment; inoculation regimen; ingredients used in combination or coincident with the specific compound, biologic or composition; the age, body weight, sex, diet, physiology and general health of the subject being treated; and like factors well known to one of skill in the relevant scientific art. Some variation in dosage or amounts will necessarily occur depending upon the condition of the subject being treated.

As used herein, the term “administering” refers to providing an effective amount of the present composition to a subject via any of a number of potential delivery mechanisms, including, but not limited to, powders that may be mixed with aqueous solutions and taken orally and/or the like. The composition of the present invention can be administered individually but may also be administered with other compounds, excipients, fillers, binders, carriers, or other vehicles selected based on the chosen route of administration and standard pharmaceutical practice. Administration may be by way of carriers or vehicles, such as injectable solutions, including sterile and non-sterile aqueous or non-aqueous solutions, or saline solutions; multi-phase systems; capsules; tablets; granules; pellets; powders; suspensions, emulsions, or microemulsions; patches; micelles; lipo-somes; vesicles; implants, including microimplants; synthetic polymers; microspheres; nanoparticles; and the like.

In one of its broadest aspects, the invention embodies a process that comprises combining one or more plant-based ingredients, natural extracts, polyphenolic compounds, yeast, vitamins, minerals, and fats to provide a composition generally intended to be administered orally to support and promote a healthy heart, cardiovascular system, and immune system, which are integrated systems. The composition in one exemplary embodiment may be combined and packaged in powdered form to be mixed with other ingredients, such as an aqueous or non-aqueous solution, and ingested orally by the subject. In an alternative embodiment, the composition may be administered orally through a carrier or vehicle, such as a capsule or pellet.

In a first aspect, the invention composition is designed to focus on the support of the subject's heart and cardiovascular system. The composition may comprise one or more plant-based ingredients, extracts, and oils that target and reduce LDL cholesterol levels of the subject and improve cardiovascular risk factors by improving FMD levels while reducing the systolic BP of the subject and improving hyperglycemia and hyperlipidemia in subjects who have diabetes or prediabetes. The extracts improve the subject's blood lipids, blood pressure, and endothelial functions, and the levels of blood glucose and glycosylated hemoglobin in the subject. The composition may include one or more polyphenolic compounds that reduce the subject's systolic blood pressure, as well as the subject's triglyceride plasma levels and cholesterol, and increase the antioxidant indicia of the subject.

The composition may comprise, without limitation, psyllium, tomato extract, fenugreek extract, resveratrol, red yeast rice extract, and medium chain triglyceride (MCT) oil in various amounts. In one exemplary embodiment, the composition may comprise psyllium up to about 4 grams (g), a 4:1 ratio amount of tomato extract up to about 30 milligrams (mg), which is the equivalent of about 1.2 grams of tomato, a 10:1 ratio amount of fenugreek extract up to about 30 milligrams (an equivalent of about 300 milligrams of whole fenugreek), resveratrol up to about 25 milligrams, a 4:1 ratio amount of red yeast rice extract (Monascus purpureus seed) up to about 150 milligrams (an equivalent of about 600 milligrams of red yeast rice), and medium chain triglyceride (MCT) oil up to about 4 grams.

An example composition is as follows.

TABLE 1
Composition for Heart and Cardiovascular System

| Ingredients | Amount (Up to) | |
|---|---|---|
| Psyllium | 4 | g |
| Tomato Extract | 120 | mg |
| Fenugreek Extract | 300 | mg |
| Resveratrol | 25 | mg |
| Red Yeast Rice Extract | 600 | mg |
| MCT | 4 | g |

In a second aspect, the invention composition is designed to focus on the support of the subject's immune system. The composition may comprise one or more vitamins, minerals, and plant-based ingredients that, when combined, support a subject's immune system by targeting the subject's respiratory system, cell-mediated immune response, and cellular counts of CD3, CD4, and CD8. For instance, the composition may include one or more vitamins that, when taken, lower the occurrence of illnesses, reduce the duration of respiratory tract infections, including acute respiratory infections, and increase CD3, CD4, and CD8 levels while reducing CRP and hsCRP levels. The composition also increases DTH and antibody levels and enhances T-cell functions in addition to alpha-tocopherol content in plasma and PBMC's, increasing antigen responses to DTH. The composition includes plant-based ingredients that include anti-oxidative and anti-inflammatory properties.

The composition may comprise, without limitation, vitamins C, D3, E, and A, zinc, selenium (as selenomethionine), and Myrciaria dubai or organic camu camu fruit powder. In one exemplary embodiment, the composition may comprise up to about 437 milligrams of vitamin C, up to about 25 micrograms of vitamin D3, up to about 555 micrograms of vitamin A, up to about 15 milligrams of zinc, up to about 20 micrograms of selenium, up to about 83 milligrams of vitamin E, and up to about 250 milligrams of Myrciaria dubia.

An example composition is as follows.

TABLE 2
Composition for Immune System

| Ingredients | Amount (Up to) | |
|---|---|---|
| Vitamin C | 437 | mg |
| Vitamin D3 | 25 | mcg |
| Vitamin A | 555 | mcg |
| Zinc | 15 | mg |
| Selenium | 20 | mg |
| Vitamin E | 83 | mg |
| Organic Camu Camu | 250 | mg |

In another exemplary embodiment, the composition may comprise, without limitation, psyllium up to about 4 grams (g), a 4:1 ratio amount of tomato extract up to about 30 milligrams (mg), which is the equivalent of about 120 milligrams of tomato, a 10:1 ratio amount of fenugreek extract up to about 30 milligrams (an equivalent of about 300 milligrams of whole fenugreek), resveratrol up to about 25 milligrams, red yeast rice extract (monascus purpureus seed) up to about 150 milligrams (an equivalent of about 600 milligrams of red yeast rice), and medium chain triglyceride (MCT) oil up to about 4 grams, up to about 437 milligrams of vitamin C, up to about 25 micrograms of vitamin D3, up to about 555 micrograms of vitamin A, up to about 15 milligrams of zinc, up to about 20 micrograms of selenium, up to about 83 milligrams of vitamin E, and up to about 250 milligrams of Myrciaria dubia.

TABLE 3
Composition for Immune and Cardiovascular System

| Ingredients | Amount (Up to) | |
|---|---|---|
| Psyllium | 4 | g |
| Tomato Extract | 120 | mg |
| Fenugreek Extract | 300 | mg |
| Resveratrol | 25 | mg |
| Red Yeast Rice Extract | 600 | mg |
| MCT | 4 | g |
| Vitamin C | 473 | mg |
| Vitamin D3 | 25 | mcg |
| Vitamin A | 555 | mcg |
| Zinc | 15 | mg |
| Selenium | 20 | mg |
| Vitamin E | 83 | mg |
| Organic Camu Camu | 250 | mg |

In one non-limiting example, a method of use of the composition may include the steps of adding approximately 15 to 20 fl oz of the composition to an aqueous solution, such as water, or a non-aqueous solution, such as milk. Once added, the composition and solution are thoroughly blended together. In one exemplary embodiment, mixing the composition with the solution may be done by shaking the mixture until any solid components (e.g., clumping of the composition within the solution) are broken down and smooth. Alternatively, a blender or frother may be used to blend the composition with the solution until the mixture is smooth. The mixture may be chilled or served over ice, as preferred, for consumption. Although the subject may consume the composition at any time, the mixture should be consumed in the morning or early afternoon for best results. The composition may also serve as a meal replacement. The composition does not include any artificial ingredients, preservatives, added sugars, caffeine, gluten, soy, or gmos.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

What is claimed is:

1. A composition for supporting a subject's integrated health system, comprising, psyllium, tomato extract, fenugreek extract, resveratrol, red yeast rice extract, and medium chain triglyceride (MCT) oil.

2. The composition of claim 1, wherein the composition includes an effective amount of:
up to about 4 grams of psyllium,
up to about 120 milligrams of tomato extract,
up to about 300 milligrams of fenugreek extract,
up to about 25 milligrams of resveratrol,
up to about 600 milligrams of red yeast rice extract, and
up to about 4 grams of medium chain triglyceride oil.

3. The composition of claim 1, further comprising an aqueous or non-aqueous solution.

4. The composition of claim 1, wherein the composition includes an effective amount of:
about 4 grams of psyllium,
about 120 milligrams of tomato extract,
about 300 milligrams of fenugreek extract,
about 25 milligrams of resveratrol,
about 600 milligrams of red yeast rice extract, and
about 4 grams of medium chain triglyceride oil.

5. The composition of claim 1, wherein the composition consists of an effective amount of:
4 grams of psyllium,
120 milligrams of tomato extract,
300 milligrams of fenugreek extract,
25 milligrams of resveratrol,
600 milligrams of red yeast rice extract, and
4 grams of medium chain triglyceride.

6. The composition of claim 1, further comprising vitamin C, vitamin D3, vitamin E, vitamin A, zinc, selenium, and camu camu fruit powder.

7. The composition of claim 6, wherein the composition includes an effective amount of:
up to about 437 grams of vitamin C,
up to about 25 micrograms of vitamin D3,
up to about 555 micrograms of vitamin A,
up to about 15 milligrams of zinc,
up to about 20 micro grams of selenium,
up to about 83 milligrams of vitamin E, and
up to about 250 milligrams of Myrciaria dubia.

8. The composition of claim 6, wherein the composition includes an effective amount of:
about 437 grams of vitamin C,
about 25 micrograms of vitamin D3,
about 555 micrograms of vitamin A,
about 15 milligrams of zinc,
about 20 micro grams of selenium,
about 83 milligrams of vitamin E, and
about 250 milligrams of Myrciaria dubia.

9. The composition of claim 6, wherein the composition consists of an effective amount of:
437 grams of vitamin C,
25 micrograms of vitamin D3,
555 micrograms of vitamin A,
15 milligrams of zinc,
20 micrograms of selenium,
83 milligrams of vitamin E, and
250 milligrams of Myrciaria dubia.

10. A composition for supporting a subject's integrated health system, comprising, vitamin C, vitamin D3, vitamin E, vitamin A, zinc, selenium, and camu camu fruit powder.

11. The composition of claim 10, wherein the composition includes an effective amount of:
up to about 473 grams of vitamin C,
up to about 25 micrograms of vitamin D3,
up to about 555 micrograms of vitamin A,
up to about 15 milligrams of zinc,
up to about 20 micro grams of selenium,
up to about 83 milligrams of vitamin E, and
up to about 250 milligrams of Myrciaria dubia.

12. The composition of claim 10, wherein the composition includes an effective amount of:
about 437 grams of vitamin C,
about 25 micrograms of vitamin D3,
about 555 micrograms of vitamin A,
about 15 milligrams of zinc,
about 20 micro grams of selenium,
about 83 milligrams of vitamin E, and
about 250 milligrams of Myrciaria dubia.

13. The composition of claim 10, wherein the composition consists of an effective amount of:
437 grams of vitamin C,
25 micrograms of vitamin D3,
555 micrograms of vitamin A,
15 milligrams of zinc,
20 micro grams of selenium,
83 milligrams of vitamin E, and
250 milligrams of Myrciaria dubia.

14. A method for providing a consumable composition designed to support a subject's integrated health system, comprising the steps of,
providing a composition comprising psyllium, flaxseed, tomato extract, fenugreek extract, resveratrol, red yeast rice extract, medium chain triglyceride (MCT) oil, vitamin C, vitamin D3, vitamin E, vitamin A, zinc, selenium, and camu camu fruit powder;
adding the composition to an aqueous or non-aqueous solution; and
blending the aqueous or the non-aqueous solution with the composition.

15. The method of claim 14, wherein the aqueous or the non-aqueous solution is within a range of about 15 to 20 fl oz.

16. The method of claim 14, wherein the composition includes an effective amount of:
up to about 4 grams of psyllium,
up to about 120 milligrams of tomato extract,
up to about 300 milligrams of fenugreek extract,
up to about 25 milligrams of resveratrol,
up to about 600 milligrams of red yeast rice extract,
up to about 4 grams of medium chain triglyceride oil,
up to about 437 grams of vitamin C,
up to about 25 micrograms of vitamin D3,
up to about 555 micrograms of vitamin A,
up to about 15 milligrams of zinc,
up to about 20 micro grams of selenium,
up to about 83 milligrams of vitamin E, and
up to about 250 milligrams of Myrciaria dubia.

17. The method of claim 14, wherein the composition includes an effective amount of:
about 4 grams of psyllium,
about 120 milligrams of tomato extract,
about 300 milligrams of fenugreek extract,
about 25 milligrams of resveratrol,
about 600 milligrams of red yeast rice extract,
about 4 grams of medium chain triglyceride oil,
about 437 grams of vitamin C,
about 25 micrograms of vitamin D3,
about 555 micrograms of vitamin A,
about 15 milligrams of zinc,
about 20 micro grams of selenium,
about 83 milligrams of vitamin E, and
about 250 milligrams of Myrciaria dubia.

18. The method of claim 14, wherein the composition consists of an effective amount of:
4 grams of psyllium,
120 milligrams of tomato extract,
300 milligrams of fenugreek extract,
25 milligrams of resveratrol,
150 milligrams of red yeast rice extract,
4 grams of medium chain triglyceride oil,
437 grams of vitamin C,
25 micrograms of vitamin D3,
555 micrograms of vitamin A,
15 milligrams of zinc,
20 micro grams of selenium,
83 milligrams of vitamin E, and
250 milligrams of Myrciaria dubia.
